(19) United States
(12) Patent Application Publication
(10) Pub. No.: US 2010/0114138 A1
(43) Pub. Date: May 6, 2010

(54) Nerve Decompression Scissors

(76) Inventor: Michael Graham, Shelby Township, MI (US)

Correspondence Address: KILE GOEKJIAN REED & MCMANUS, 1200 NEW HAMPSHIRE AVE, NW, SUITE 570, WASHINGTON, DC, 20036, US

(21) Appl. No.: 12/574,699

(22) Filed: Oct. 6, 2009

Related U.S. Application Data
(60) Provisional application No. 61/102,930, filed on Oct. 6, 2008.

(51) Int. Cl. A61B 17/32

(52) U.S. Cl. 606/174

(57) ABSTRACT

A surgical instrument and method for freeing tissue in a nerve decompression operation. Tissue is freed from the area surrounding a nerve with use of the blunt tip on one jaw of the instrument. The freed tissue is then cut with the bladed region of the instrument and removed from the area surrounding the nerve while leaving the nerve untouched. The instrument includes a pair of pivotally connected arms. Each of the arms is elongated and has a free end and a handle end, the free end of each having a blade, the handle end of each having a finger loop for the surgeon to grasp. One of the free ends is longer than the other, with a blunt, rounded tip and edge for freeing tissue without cutting. The pivot between the two arms separates the two sections and allows the arms to move with respect to each other within a plane of pivotal movement so that the tissue is selectively cut along the extent of the overlap of the arms.

### BACKGROUND OF THE INVENTION

This invention generally relates to surgical instruments, and more specifically to a surgical instrument designed to selectively cut fibers during a nerve decompression operation. The instrument is particularly suited to decompress the posterior tibial nerve on the inner ankle.

A common condition experienced by patients is a numbness or pain, for example, in the ankle region. Alternatively, the patient may experience muscle weakness in this area or trouble maintaining balance. One common cause underlying these symptoms is nerve damage due to compression of the nerve. Typically, the nerve is compressed by tissue surrounding the nerve which has become inflamed and swollen. Whatever the cause, the pressure on the nerve must be relived or permanent nerve damage could result. In the ankle region, one common concern is Tarsal Tunnel Syndrome. This is caused by compression of the posterior tibial nerve by the lacinate ligament.

Nerve decompression surgery is one treatment to treat nerve compression. In the case of the posterior tibial nerve, the lacinate ligament, which passes above the nerve and presses down upon it, is in the region passing over the nerve. Once the ligament is severed, it retracts from the site of the cut and the pressure on the underlying nerve is relieved.

One critical aspect of nerve decompression surgery is that the surrounding tissue must be removed without damaging any nerve fibers. This is particularly difficult in the case of the cutting the lacinate ligament because it is a blind operation. The ligament passing above the nerve obscures the nerve from the surgeon's view.

Different surgical techniques are employed to separate and cut tissue from around nerve fibers. Some surgical techniques rely heavily on a surgeons skillful use of a scalpel to precisely cut away only surrounding tissue without also cutting into any nervous tissue. This has the disadvantage of requiring a very steady hand and being painstakingly slow. Moreover, there is the ever present danger of an inadvertently nick of the nerve fibers if either the scalpel or the target tissues are jostled even a small amount.

Alternatively, a surgeon can use a blunt instrument, such as a surgical probe or a forceps to separate the nerve fibers from the surrounding tissue. While this has the advantage that the blunt edge is unlikely to nick nervous tissue, there is a disadvantage. The use of two surgical instruments requires a larger incision. This in turn inevitably leads to a more damage to surrounding tissues and thus longer recovery times and more frequent post operative complications. Also, a two handed surgical procedure is more difficult. The surgeon must simultaneously coordinate the movement of both hands.

In the case of the cutting of the lacinate ligament, there is the additional problem of wound dehiscence, the opening of a wound (or the failure of a wound to properly heal) due to skin tension which pulls the two halves of the wound apart. Skin tension is particularly high in the ankle region. Accordingly, there is a long felt need for surgeons to make the smallest incision possible for cutting the lacinate ligament so as to keep the risk of wound dehiscence as small as possible.

Microsurgical techniques can be employed. While these methods result in extremely precise work, they require elaborate instrumentation and can take an extremely long time. The handles of these instruments must be connected to their functional ends through a complex gear system such that a macroscopic motion of the handle is translated to a microscopic motion of the functional end. Also a microscopic viewing system must be employed. This elaborate instrumentation makes the procedure difficult to perform, if not impossible, in a clinical or “out patient” setting. A state-of-the-art operating room is required.

A variety of specially shaped scissors have been developed for various purposes. One such scissors combines tissue dissection and surgical implantation. These scissors are designed to grasp and release a tissue, in one conformation, and dissect tissue in another conformation. The instrument is designed to replace methods of using both a scissors to open a pocket in layers of tissue and a tweezers, hemostats, or graspers to position and insert mesh or other material into the tissue pocket. The scissors also measures distances or lengths of dissected tissue and anatomical structure, eliminating the extra step of using a ruler. Each blade of the scissors has a flattened tip for blunt dissection and a non-cutting clamping portion for selectively grasping a surgical implant device. The blades pivot relatively freely between the dissection and clamping positions.

Another specially shaped instrument is designed for use with the optic nerve. This instrument clamps the optic nerve during the removal of a diseased eyeball. Such clamping of the nerve was formerly done by feel with a hemostat, which resulted in uncertainty as to the extent of tissue secured in the clamp. The optic nerve clamp has a specific size and curvature designed to reduce the loss of tissue behind the eye by facilitating isolation and clamping of the optic nerve alone and can also reduce the incidence of unintentional excisions in the back part of the eyeball and other damage to the surrounding tissue. The clamp includes a pair of jaws which are curved, with one tip shorter than the other. The end of the longer tip is tapered. The varied length in the tip portions is adapted to allow the optic nerve to be located between the ends of the shorter and longer tips. The longer tip has a smooth flat surface adjacent to the tip. The variation in tip lengths allows the surgeon to slide the instrument as needed to precisely locate the nerve before clamping it. It is also suggested that two clamps with opposite curvatures could be created, one for the left eye and one for the right. This instrument differs from the disclosed invention. For example, this instrument does not include a scissors function.

Another instrument is designed to cut one layer of tissue while leaving an underlying tissue layer unscathed. This design relies primarily on a sharp right angle turn in the tip of the scissors which allows the surgeon to cut the target tissue at an oblique angle.

None of these instruments are able to perform a nerve compression operation. They lack the ability to both separate the surrounding tissue from the nerve and dissect away the surrounding tissue.

### SUMMARY OF THE INVENTION

Disclosed herein is a surgical instrument that can perform multiple surgical tasks and requires the use of only one hand. The decompression scissors can (1) free a target tissue to be removed from nerve fiber and (2) cut away and remove the target tissue without damaging the nerve tissue.

The decompression scissors includes a pair of arms connected at a pivot point. The scissors have two arms each of which have a jaw on one side of a pivot and a handle end on the opposite side of the pivot. The portion of the longer jaw extending beyond the shorter jaw has a blunt, rounded tip and no blade along the inside edge. The portion of the longer jaws that overlaps with the shorter jaw is bladed.

In an additional embodiment of the present invention, the jaws of both arms can be curve upwards, within the plane containing the arms and handles of the scissors.

### BRIEF DESCRIPTION OF THE DRAWINGS

FIG. 1 illustrates a perspective view of a pair of scissors within the scope of the invention disclosed.

FIG. 2 is a curved embodiment of the scissors.

FIG. 3 is a close up of the scissors' jaws showing the bladed edges.

FIGS. 4-7 illustrate a method of decompression of the posterior tibial nerve.

### DETAILED DESCRIPTION OF PREFERRED EMBODIMENTS

### The Instrument

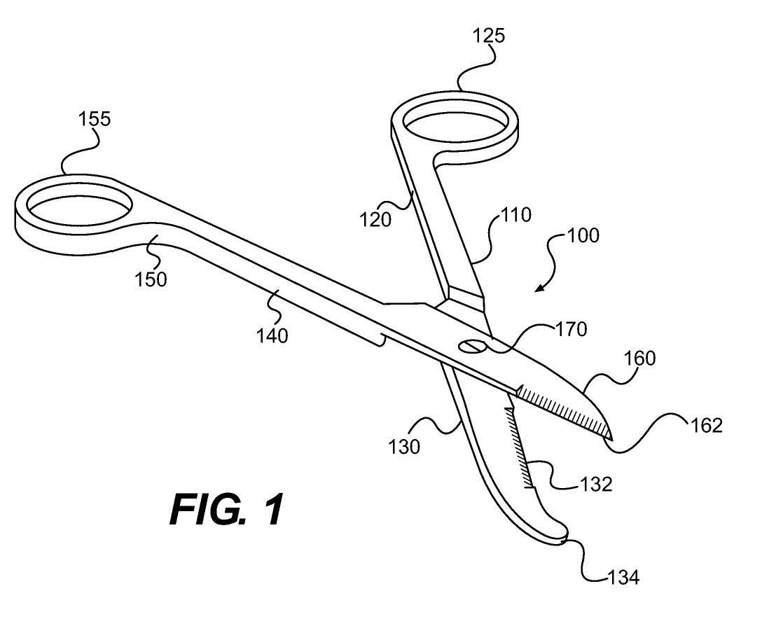

FIG. 1 illustrates a perspective view of a surgical instrument 100 within the scope of the invention. The invention comprises a first arm 110 which is divided at a pivot point 140 into a handle on one side 120 and a jaw region 130 at the other side. A second arm 140 is divided at the pivot point 140 into a handle on one side 150 and a jaw region 150 on the other side.

The jaw region of the first arm comprise a bladed area 132 and a tip 134. The tip is specially designed so as to separate nervous tissue from surrounding tissue. One embodiment within the scope of the invention is a blunt tip 134 as shown in FIG. 1. The blunt tip can also be curved upward as shown 134. When the scissors are slid into place, the curve encourages the nerve to pass below the jaw and not become trapped between the jaws.

The tip of this jaw 134 can take any of several shapes, including both rounded surfaces, flattened surfaces, or a combination of both rounded and flattened surfaces. For example the upper, inner surface of the tip can be flattened so that this surface will travel closely along the underside of the lacinate ligament when used as described below in a method of decompression of the posterior tibial nerve.

The second first arm 140 has a jaw region 150 designed to interact with the corresponding region of the first arm. The bladed region 152 is typically designed to be longitudinally coextensive with the bladed region of the corresponding bladed region on the first arm. The jaw region typically ends with a sharp tip. The scissors will function, however, with a wide variety of common shapes at the end of the jaw region of the second arm.

The opposite sides of the first and second arms contain the handles 125, 155. The handles can be any shape or size which is suitable for grasping and operating the scissors.

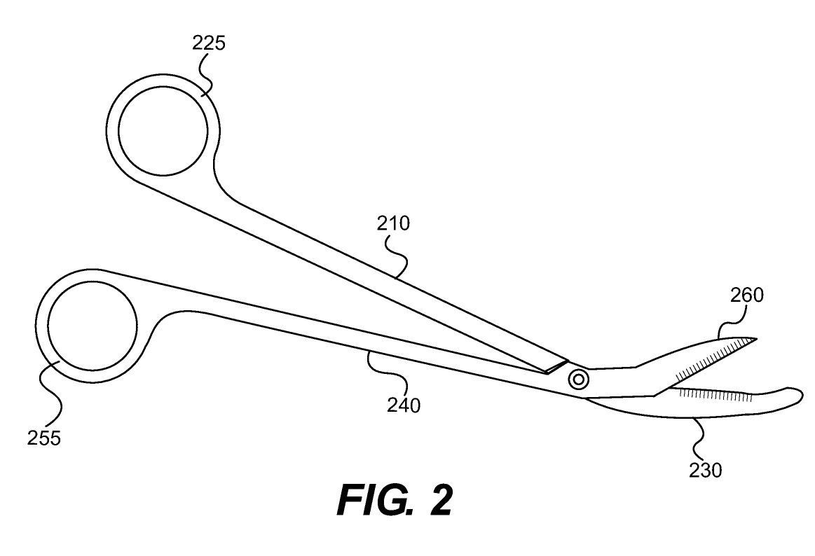

FIG. 2 illustrates another preferred embodiment of the invention. The jaw region is given a concave curve in the plane of the scissors as shown. The invention is operable over a wide range of curvatures. The exact degree will depend on the particular nerve and the particular location of the decompression operation.

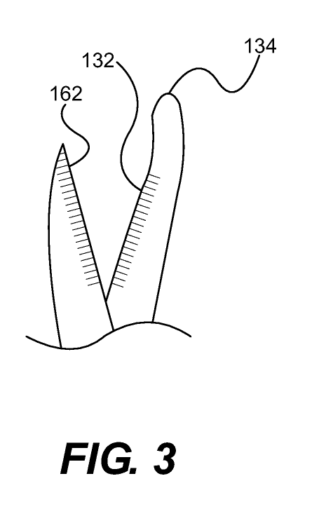

FIG. 3 is a close up of the jaw region. Illustrated are preferred bladed regions 132, 152; the blunt tip 134.

The relative lengths of the handle region and the jaw regions can be varied depending on the amount of leverage the surgeon need to generate. The optimal ratio of the lengths of the handle region versus the jaw region will vary depending on the material used and the toughness of the tissue to be cut.

Similarly the overall length of both the instrument, as well as each component of the instrument will depend on the particular body location and the particular tissue(s) involved in the nerve decompression operation.

### The Method

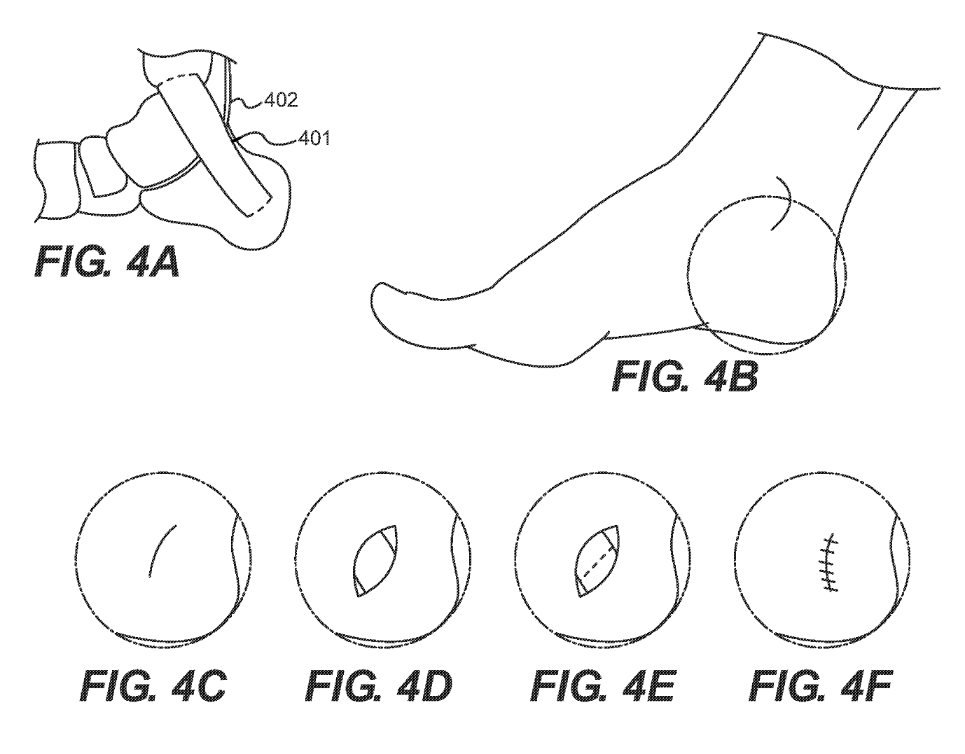

FIGS. 4a-f illustrate a method of nerve decompression using an instrument within the scope of the invention. In FIG. 4a, the ankle structures are simplified so as to highlight the lacinate ligament 401 and the posterior tibial nerve 402. This particular nerve is located in the inner ankle region as shown in FIG. 4b. The method comprises: (1) making a small incision in the inner ankle region as shown in FIG. 4c, (2) exposing the lacinate ligament as shown in FIG. 4d, (3) cutting the ligament along the dotted line as shown in FIG. 4e. This is accomplished by positioning the scissors, described above, so that the lacinate ligament is between the jaws of the scissors. As the scissors are slid into place, the customized shape of the lower jaw keeps the posterior tibial nerve from becoming trapped between the jaws of the scissors. This is very important because the surgeon will be unable to see this nerve. The lower jaw tip will separate the nerve from the ligament above and the nerve will pass safely below the lower jaw of the scissors. After the ligament is cut, the ends of the ligament will retract thus releasing the pressure on the underlying nerve. Finally, the incision is closed as shown in FIG. 4f. As stated earlier, the smaller the incision the better. For several reasons this incision will be smaller when employing the above method. This is because only a single surgical instrument is used during the cutting of the lacinate ligament. Moreover, because the surgeon can be confident that the posterior tibial nerve will not be cut, there is no need to widen the incision in order to view the location of the nerve.

In describing the invention, reference is made to preferred embodiments. Those skilled in the art and familiar with the disclosure of the subject invention, however, will recognize additions, deletions, substitutions, modifications and/or other changes which will fall within the purview of the invention as defined in the following claims.

1. A surgical instrument for decompressing nerve fibers by excising tissue adjacent to said nerve fibers comprising:
two elongated arms,
said first arm comprising,
a first jaw region which further comprises a sharp bladed region for cutting tissue and longitudinally coextensive with the bladed region of a second jaw section, and,
a distal region adjacent to said sharp bladed region comprising a blunt tip, and
a first handle region separated from said first jaw region by a pivot point, which further comprises a structure for grasping and manipulation by a surgeon
said second arm comprising,
a second jaw region which further comprises a sharp bladed region for cutting tissue, and
a second handle section, separated from said first jaw region by a second pivot point, which further comprises a structure for grasping and manipulation by a surgeon; and
said first arm and said second arm are connected at a pivot point,
wherein, said blunt tip is configured for and operable to separate tissue adjacent to nerve fibers from the nerve fibers and said blade sections are configured to cut and remove said adjacent tissue.

2. A surgical instrument for decompressing nerves as defined in claim 1, wherein said first and second jaw sections have a convex curve in the plane of the scissors.

3. A surgical instrument for decompressing nerves as defined in claim 1, wherein said blunt tip region has a cross sectional shape selected from the group consisting of round, oval, round with flattened upper and lower portions and oval with flattened upper and lower portions.

4. A method of decompressing nerves comprising:
making an incision above the nerve to be decompressed,
exposing a tissue to be removed;
inserting a decompression scissors so that the tissue to be removed is between the jaws of the scissors but the nerve is not between the jaws,
cut the tissue to be removed, and
close the incision,
wherein said decompression scissors comprises:
two elongated arms,
said first arm comprising,
a first jaw region which further comprises a sharp bladed region for cutting tissue and longitudinally coextensive with the bladed region of a second jaw section, and,
a distal region adjacent to said sharp bladed region comprising a blunt tip, and
a first handle region separated from said first jaw region by a pivot point, which further comprises a structure for grasping and manipulation by a surgeon
said second arm comprising,
a second jaw region which further comprises a sharp bladed region for cutting tissue, and
a second handle section, separated from said first jaw region by a second pivot point, which further comprises a structure for grasping and manipulation by a surgeon; and
said first arm and said second arm are connected at a pivot point,
wherein, said blunt tip is configured for and operable to separate tissue adjacent to nerve fibers from the nerve fibers and said blade sections are configured to cut and remove said adjacent tissue.

5. The method of claim 4 wherein said decompressed nerve is the posterior tibial nerve.

6. The method as defined in claim 5 wherein said incision has a length specifically designed to minimize the probability of wound dehiscence.

7. The method as defined in claim 6 wherein said incision length is large enough to allow cutting a lacinate ligament but not large enough to expose the tibial nerve.
